(19) United States
(12) Patent Application Publication
(10) Pub. No.: US 2021/0260334 A1
(43) Pub. Date: Aug. 26, 2021

(54) BRAIN DRUG DELIVERY SYSTEM AND METHOD

(71) Applicant: Postsurgical Therapeutics, Inc., Costa Mesa, CA (US)

(72) Inventor: Soonkap Hahn, Irvine, CA (US)

(21) Appl. No.: 17/180,611

(22) Filed: Feb. 19, 2021

Related U.S. Application Data
(60) Provisional application No. 62/979,107, filed on Feb. 20, 2020.

(51) Int. Cl. A61M 25/00; A61M 25/01

(52) U.S. Cl. CPC A61M 25/0026; A61M 25/0084; A61M 2210/0693; A61M 2202/064; A61M 2205/02; A61M 25/0108

(57) ABSTRACT

A brain drug delivery system having a catheter and a delivery assembly is provided. The catheter has a first channel with a first exit hole and a second channel with a second exit hole, both the first exit hole and the second exit hole being disposed at a distal end of the catheter, the second exit hole being adapted such that to cause a sideways turn of a movable inner plastic tube exiting therethrough. The catheter also being adapted to house a delivery assembly selectively within the first and second channel. While the delivery assembly has the movable inner plastic tube with a tube exit hole and an image-guided plunger being adapted to penetrate the movable inner plastic tube and push a powder form of drug microparticles via the tube exit hole.

### CROSS-REFERENCE TO RELATED APPLICATIONS

This application claims the benefit of U.S. Provisional Application No. 62/979,107, filed Feb. 20, 2020, which is hereby incorporated by reference, to the extent that it is not conflicting with the present application.

### BACKGROUND OF INVENTION

### 1. Field of the Invention

The invention relates generally to therapeutic systems and methods and more specifically to systems and methods for improved drug delivery into the brain.

### 2. Description of the Related Art

Diseases associated with the brain are very difficult to treat. One of the main reasons for this difficulty comes from the blood-brain barrier (BBB). The BBB is a specialized structure consisting of brain blood vessels and capillary endothelial cells that forms tight junctions. These tight junctions limit the transport of various molecules into the brain. The BBB is a defense mechanism that prohibits unwanted, harmful materials entering into the brain. However, this defense mechanism imposes a very difficult task on developing drugs that treat brain diseases effectively. About 98% of small molecule drugs and almost all of large molecular biologics do not pass through the BBB when taken by two most common routes, orally and intravenously. This is why developing effective drugs for treating the brain diseases is very difficult.

One way to overcome the BBB is to make a small hole into the skull and deliver a drug solution through a catheter directly into the brain. This method enables bypassing the BBB. However, inside the brain there is a relatively high pressure (intracranial pressure between 5 and 15 mmHg). This high pressure hinders the diffusion of delivered drug by limiting the diffusion of the drug into the treated brain tissue typically to less than 3 mm (millimeters). This limits the drug's overall efficacy.

In order to improve the diffusion, convection enhanced delivery (CED) method has been actively developed. The CED method also uses the catheter but simultaneously generates a pressure gradient at the tip of a catheter by a motor-driven pump to push the drug solution through the interstitial space of the brain. This method bypasses the BBB as well as improves the diffusion of delivered drug to up to 2-3 cm (centimeters) deep into the treated brain tissue.

It appears that 5 (five) different types of CED have been developed: 1) end port cannula, 2) multi-port cannula, 3) micro porous tipped cannula, 4) balloon tipped cannula, and 5) stepped profile cannula. However, these CED methods possess some shortcoming such as backflow of delivered drug solution (reflux) which can lead to leakage into unintended areas of the brain and cause some toxicity therein. The reflux also decreases the volume and the predictability of the drug distribution to the intended areas of the brain.

The reflux is closely related to the infusion rate. In order to avoid the reflux, the infusion rate must be kept slow. This slow infusion rate requires a long infusion time of hours to days, which is cumbersome and also increases the incidence of infection. In addition, the infused drug solution may flow into an area not intended for treatment, which may cause some serious toxicity (i.e., cytotoxic agents) in that unintended area. Since the infused drug is degraded or removed from the brain in a relatively short time (i.e., within a day or so), in part due to the fact that the drug is administered in a solution form, either an indwelling catheter for a long time or a frequent installment of catheter is required to prolong the drug treatment. Again, the indwelling catheter and the frequent installment may increase the incidence of infection. The CED methods are also cumbersome to use.

Therefore, for all of the above shortcomings of the existing systems and methods, there is still a need for a new and improved method and system for drug delivery into the brain that overcome many of the above shortcomings.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description.

In an aspect, new and improved method and system for drug delivery into the brain are provided. In an example, the system may include four components, such as a catheter, a delivery assembly, an imaging system, and a powder of hydrogel-PLGA or PLGA drug microparticles.

In an example, the catheter may consist of an open tube and a delivery assembly with a movable inner plastic tube and image-guided plunger. The open tube may have two distal holes at the distal end, one hole disposed straight at the distal end and one on the side of the tube near the distal end. The two-hole configuration of the tube can position a tip of the delivery assembly in various directions toward tissues that need drug treatment.

In an example, the delivery assembly consists of a movable inner plastic tube and image-guided plunger and can be guided by an imaging system such as ultrasound, magnetic resonance imaging (MRI) or other devices. The tip of the plunger in the delivery assembly can be positioned precisely by an imaging system to the tissues necessary for drug treatment.

In an aspect, a powder form of hydrogel-PLGA or PLGA drug microparticles can be filled into the movable inner plastic tube in the delivery assembly and delivered by for example a plunger to the tissues that need treatment. This delivery procedure can be repeated multiple times during a single drug administration intervention, in order to deliver the powder over the entire tissues where treatment is necessary. In an example, the hydrogel-PLGA or PLGA drug microparticles can provide a sustained, controlled release of encapsulated drug(s) over 1-12 weeks.

Thus, an advantage of the disclosed method and system for drug delivery into the brain is that it eliminates or reduces the need for prolonged or repeated drug delivery interventions, which makes the disclosed system and method more efficient (less time and cost), more effective in treating the targeted brain tissue and less prone to infections. Another advantage is that, during administration, the system and method disclosed can deliver drugs quickly and precisely to the tissues in need of treatment. Another advantage is that the reflux described above is avoided, because of the powder form of the hydrogel-PLGA or PLGA drug microparticles, which enable the microparticles to stick to the treated tissue. In the case of the hydrogel-PLGA drug microparticles, the drug microparticles can also swell (due to the presence of the hydrogel component in the microparticles) to further secure them to the treated tissue.

As another advantage, using the disclosed system and method, the shortcoming of the existing methods, characterized by the drug diffusion being hindered due to the high pressure in the brain, can be overcome by administering the drug powder to multiple spots within the diseased tissue, whereby one spot is close to the next spot (i.e., less than 1 cm).

The above aspects, examples and advantages, as well as other aspects, examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 illustrates a cross-sectional view of a new and improved catheter drug delivery system, according to an aspect.

FIG. 2A illustrates a side view of a delivery assembly, according to an aspect.

FIG. 2B illustrates a side view of a movable inner plastic tube, according to an aspect.

FIG. 2C illustrates a side view of an image-guided plunger, according to an aspect.

FIG. 3 illustrates a perspective view of a new and improved catheter drug delivery system, according to an aspect.

FIG. 4A illustrates a side view of a new and improved catheter drug delivery system with a retracted delivery assembly, according to an aspect.

FIG. 4B illustrates a side view of a new and improved catheter drug delivery system with the delivery assembly protruding, according to an aspect.

FIG. 5A illustrates a front view of the tip of the delivery assembly pushed into tumor tissue, according to an aspect.

FIG. 5B illustrates a front view of a plunger reinserted into the movable inner plastic tube and pushed to deliver the powder into the tumor tissue, according to an aspect.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention.

It should be understood that, for clarity of the drawings and of the specification, some or all details about some structural components or steps that are known in the art are not shown or described if they are not necessary for the invention to be understood by one of ordinary skills in the art.

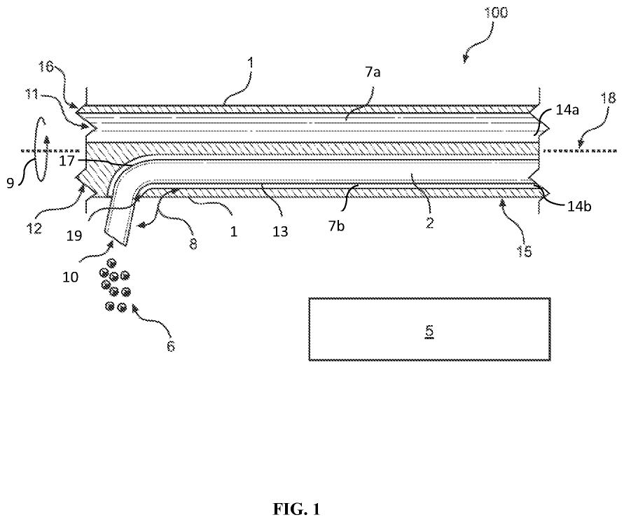

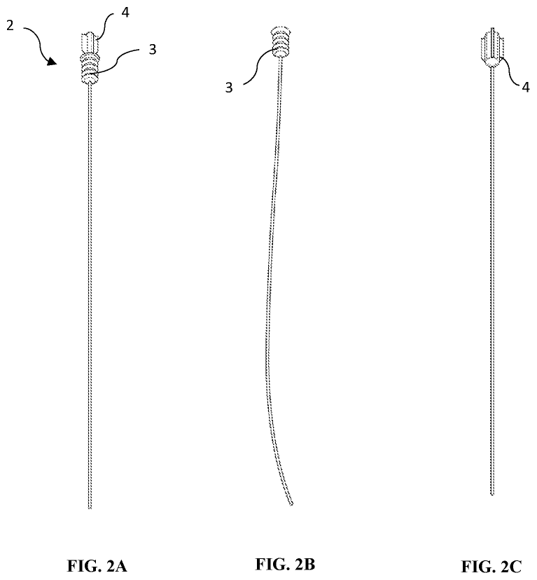

FIG. 1 illustrates a cross-sectional view of a new and improved catheter drug delivery system (“catheter-delivery assembly”) 100, according to an aspect. As shown, in an example, the system having a catheter 1 that may be made of various polymers, such as silicone, polyurethane, latex and other thermoplastic elastomers. The catheter 1 having a channeled interior and a proximal end 15 and a distal end 12 is provided. The catheter 1 may have dual channels, such as a first channel 14a and a second channel 14b, each with hollow core 7a, 7b, respectively. The channels 14a, 14b may be defined by their hollow cores 7a, 7b and their exterior walls 13, 16, as shown in FIG. 1. Additionally, the catheter 1 may have an opening (“first exit hole”) 11 at the distal end 12 of the catheter 1 and a side opening (“second exit hole”) 19 on the side of the catheter 1 and also near the distal end 12. The side opening 19, as shown, is another opening allowing a delivery assembly 2 to exit, which will be discussed in more details when referring to FIG. 2. The side opening 19 is preferably made an obtuse angle, preferably at 120-150 degrees relative to the side of the catheter 1. This may be achieved by having the entire wall 13 of the second channel 14b slanted at preferably 120-150 degrees. In another example, the second channel 14b may have a guide (not shown) near the second exit hole 19 to allow the delivery assembly 2 to curve at the selected angle. This configuration of the side opening 19 facilitates orienting a portion of the distal end 12 of a delivery assembly 2 with a movable inner plastic tube (“tube”) 3 and image-guided plunger 4 that exits the side opening 19 and an obtuse angle 8 (120-150 degrees), which will be discussed in more details when referring to FIG. 2. For example, the obtuse angle 8 may begin at the bending point 17, as shown in FIG. 1.

It should be noted that the length of the exiting side opening 19 may be somehow exaggerated in FIG. 1, for illustration purposes, and in any event, that the length is not constant during drug administration. Additionally, it should be understood that in practice, the distal end 12 of the delivery assembly 2 would preferably be positioned as close as possible to the catheter 1, or inside the catheter 1, especially during the rotational movement of the catheter-delivery assembly 100. This may be needed in order to avoid unnecessarily cutting or disturbing the diseased tissue during the up-and-down or rotational movements of the catheter drug delivery system 100 within the diseased tissue (“tumor,” “wound area,” “wound,” “tumor tissue”). Thus, in an example, the delivery assembly 2 would be retracted within the catheter 1, before rotation, and then, after rotation, the delivery assembly 2 may be guided through the side opening 19 to penetrate the diseased tissue in a slanted-transversal direction to various depths and deliver the powder drug therein. In another example, the delivery assembly 2 would be retracted and then guided through distal opening 11 to penetrate longitudinally to various depths within the diseased tissue and deliver the powder drug therein.

Once the tip 21 of the plunger 4 is precisely positioned as described herein, a known amount of the powder of hydrogel-PLGA or PLGA drug microparticles 6 can be filled into the movable plastic inner tube 3 and then delivered with, for example, a mechanical push mechanism using a plunger 4 or other mechanisms, such as a steam or solution pushing. Moreover, the movable plastic inner tube 3 has a tube exit hole 10 allowing the drug microparticles 6 to exit the system. The delivery process can be repeated to deliver the drug powder over the entire diseased area.

Additionally, the delivery system 2 may be inserted into a first channel 14a, which may be straight and run the length of the catheter 1. The first channel 14a may allow the delivery system 2 to be guided and inserted directly into a wound area. The delivery system 2 may also be inserted into a second channel 14b, which may lead to the side hole (“side opening,” “hole,” “opening”) 19. The second channel 14b may run the length of the catheter 1, but then may angle, for example, at a 120-degree angle, towards the side opening 19. Moreover, the second exit hole 19 may be adapted such that to cause a sideways turn of a movable inner plastic tube exiting therethrough. For example, the second channel 14b may achieve the sideways turning effect by having the opening 19 at the distal end of the catheter 1 with a guide to lead the movable plastic inner tube 3 to turn. And, as described herein, the sideways turn may be of an obtuse angle. Furthermore, the second exit hole 19 may be, for example, on the side of the catheter 1 to allow for the sideways turning effect.

The side hole 19 on the catheter 1 may allow the delivery assembly to penetrate the tumor or wound area (e.g., tumor) and deliver the drug either in a straight path using the first channel 14a or a curved path using the second channel 14b. The dual channels 14a, 14b allow for a 360-delivery of the drug. Furthermore, the delivery assembly 2 may be retracted while the catheter 1 rotates then exposed again in a new selected drug delivery location.

FIG. 2A illustrates a side view of a delivery assembly 2, according to an aspect. FIG. 2 shows a delivery assembly 100 consisting of a movable inner plastic tube 3 and image-guided plunger (“plunger”) 4. FIG. 2B illustrates a side view of a movable inner plastic tube 3, according to an aspect. And FIG. 2C illustrates a side view of an image-guided plunger 4, according to an aspect. The movable inner plastic tube 3 may be made of, for example, various polymers, such as silicone, polyurethane, latex and other thermoplastic elastomers. While the image-guided plunger 4 may be made of, for example, nitinol, an elastic alloy, or other elastic metals or alloys. Furthermore, the image-guided plunger 4 may be adapted to fit into the movable plastic tube 3 to form the delivery assembly 2. It should be noted that the overall delivery assembly 2 may be flexible enough to fit into the side opening 19 with an obtuse angle of 120-150 degree.

Furthermore, the plunger 4 in the delivery assembly 2 may be precisely guided by an imaging system 5 to the tissues, which is necessary for treatment. In an example, the disclosed brain drug delivery system 100 may use a plunger similar to an image-guided needle, which is used for tissue biopsy in combination with an ultrasound imaging system. Additionally, the image-guided plunger may be guided by an imaging system 5, such as magnetic resonance imaging (MM) or other devices. Moreover, the imaging system 5 may also be an ultrasound system (e.g., eZGuide™).

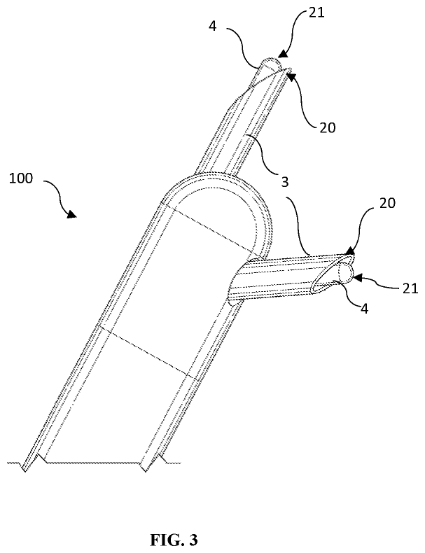

FIG. 3 illustrates a perspective view of a new and improved catheter drug delivery system 100, according to an aspect. The movable inner plastic tube 3, for example, is shaped to have a sharp edge 20 shown in FIG. 3. The sharp edge 20 of the movable inner plastic tube 3 may help the delivery assembly 2 penetrate into the tumor tissues (“wound area”). The movable inner plastic tube 3 may also protrude from the catheter to further allow the delivery assembly 2 to penetrate into the tumor tissues.

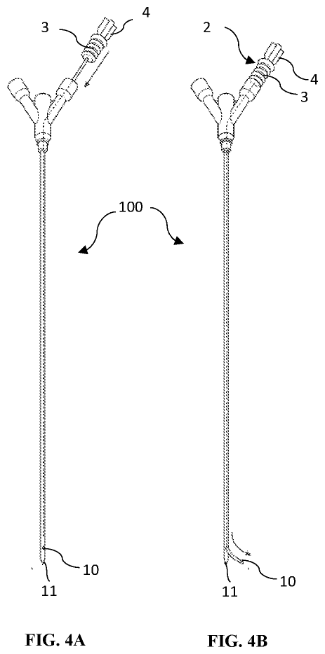

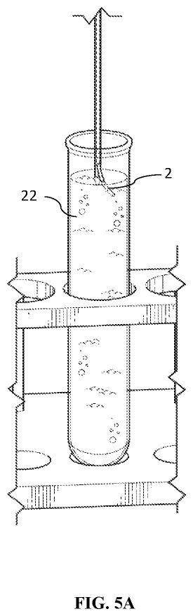

FIG. 4A illustrates a side view of a new and improved catheter drug delivery system 100 with a retracted delivery assembly 2, according to an aspect. While FIG. 4B illustrates a side view of a new and improved catheter drug delivery system 100 with the delivery assembly 2 protruding, according to an aspect. By configuring the catheter 1 as disclosed herein (using two holes 11, 19 at the end of catheter 1) and by moving the catheter-delivery assembly 100 up and down as well as rotationally, as indicated by arrow 9 and shown in FIG. 1, around the longitudinal axis 18 of the catheter 1, the delivery assembly 2 can reach the entire diseased tissue for drug administration. Thus, the drug powder 6 can be delivered to the entire diseased tissue. This can be accomplished by moving the catheter drug delivery system 100 up and down within the diseased tissue, thus accessing and delivering the drug at various depths within the diseased tissue, via a portion of the distal end 12 of the delivery system 100, which in this case would be guided to exit through the distal opening 11. Further, the drug can be delivered sideways by the catheter drug delivery system being progressively rotated up to 360 degrees while the distal end 12 of the delivery assembly is guided to exit through the slanted tube exit hole 10.

It should be noted that the obtuse angle 8 may allow the delivery assembly 2 to more likely reach all the diseased tissue and it would be easier to guide the plunger 4 and push the powder drug 6 out. Additionally, as described herein, the catheter drug delivery system 100 may allow the drugs to be delivered through the distal opening 11 in a more straight, direct path, or through the side opening 19. Furthermore, the drug 6 being able to be delivered through both the distal opening 11 and the side opening 19 allows for the wound area to be more fully treated by the drug. For example, full treatment of the wound area (i.e., a tumor) by using the catheter drug delivery system 100 to release the drugs at both the center of the wound area and the surrounding area may be critical to a patient's health. Moreover, delivering the drug through the side opening 19 may be critical to fully treat the entire wound area because of the tube exit hole 10 allowing the drug to be released 360 degrees around the wound area.

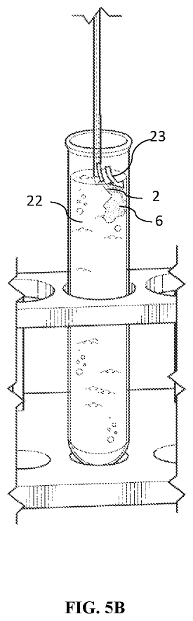

FIG. 5A illustrates a front view of the tip 21 of the delivery assembly 2 pushed into tumor tissue, according to an aspect. While FIG. 5B illustrates a front view of a plunger 4 reinserted into the movable inner plastic tube 3 and pushed to deliver the powder into the tumor tissue, according to an aspect. Additionally, FIGS. 4A, 4B, 5A, and 5B illustrate a simulation of delivering a powder using the side opening 19 with an agarose gel brain model 22 which is often used as in vitro brain model.

In this illustrated simulation, for example, a smoked paprika powder with a cherry color is used as the drug powder. First, the catheter 1 may be fitted with the delivery assembly 2. Then, the catheter drug delivery system 100 moves down to the surface of the agarose gel 22. At this point, the delivery assembly 2 is retracted inside the catheter as shown in FIG. 4A. For comparison, FIG. 4B shows the delivery assembly 2 pushed fully toward the tumor tissue. After the catheter is positioned at proper location, the tip 21 of the delivery assembly 2 is pushed into tumor tissue and positioned at predetermined spot, as shown in FIG. 5A. During positioning, the tip 21 of the delivery assembly 2 (i.e., the tip of the plunger 4) is guided by imaging system 5 (preferably ultrasound device). Then, the plunger 4 is retracted completely to leave the movable inner plastic tube 3 inside the tumor tissue. The powder is filled into the movable plastic tube 3. Then, the plunger 4 is reinserted into the movable inner plastic tube 3 and pushed, as depicted by the arrow 23, to deliver the powder into the tumor tissue as shown in FIG. 5B.

Furthermore, a method of utilizing the brain drug delivery system may begin with selecting the catheter 1 and then selectively fitting the delivery assembly 2 into the first or second channel of the catheter 1. Next, inserting the catheter with the delivery assembly into a subject and positioning the distal end of the catheter 1 in a proximity of a targeted wound area. Then, pushing the delivery assembly 2 into the wound area in the subject. And retracting the image-guided plunger 4 to begin loading drug particles 6 into the movable inner plastic tube 3. Followed by reinserting the image-guided plunger 4 into the movable inner plastic tube 3 and releasing the drug particles 6 into the wound area either straight via the first exit hole or at an angle via the second exit hole.

Hydrogel-PLGA or PLGA Drug Microparticles

The powder is preferably made of two different polymers, hydrogel and PLGA (copolymer of poly lactic acid (PLA) and poly glycolic acid (PGA)) or PLGA alone. In an example, the drug may be encapsulated into PLGA (PLGA-drug microparticles). PLGA-drug microparticles can be used directly as a treatment drug or the PLGA-drug microparticles may be embedded into biocompatible hydrogel. The hydrogel component may also contain a drug, either the same drug as in the PLGA microparticles or a different drug. The hydrogel component can prolong the release of drug encapsulated in the PLGA microparticles as well as improve their biocompatibility.

In one preferred embodiment, the hydrogel is hyaluronic acid. The resulting powder can provide a sustained, controlled release of the encapsulated drug(s) over 1-12 weeks. The long drug release mode can reduce the frequency of administration and thus, the burden of delivering drug into the brain frequently.

The duration of drug release can be controlled by for example varying the properties of PLGA and/or the degree of crosslinking of hyaluronic acid. For example, since crosslinks act as barriers, the more crosslinks are present, the harder it is for the drug released from the PLGA-drug particles to diffuse out.

Again, embedding PLGA-drug micro-particles into hyaluronic acid improves the biocompatibility of the PLGA-drug microparticles. In addition, it prolongs the release of encapsulated drug.

PLGA Microparticles

PLGA is a well-known biodegradable polymer with excellent safety profile. A number of products with a drug encapsulated in PLGA are already approved by FDA. PLGA is a copolymer of lactic acid and glycolic acid. PLGA and a drug can be fabricated in microparticles including microcapsules and microspheres. Microcapsules generally have a drug core coated with a polymer film and may be spherical or non-spherical in shape. In contrast, microspheres have drugs dispersed evenly in polymer and are spherical in shape.

PLGA microparticles are a valuable drug delivery system due to their versatility in controlling drug release rate. The drug release rate from PLGA microparticle can be controlled by adjusting a number of parameters such as 1) ratio between polylactic acid (PLA) and polyglycolic acid (PGA), 2) molecular weight and 3) size of micro-particle.

In PLGA, polylactic acid is more hydrophobic compared to polyglycolic acid and subsequently hydrolyzes (i.e., degrades) slower. For example, PLGA 50:50 (PLA:PGA) exhibits a faster degradation than PLGA 75:25 due to preferential degradation of glycolic acid proportion if two polymers have the same molecular weights. PLGA with higher molecular weight exhibits a slower degradation rate than PLGA with lower molecular weight. Molecular weight has a direct relationship with the polymer chain size. Higher molecular weight PLGA has longer polymer chain and requires more time to degrade than lower molecular weight PLGA. In addition, an increase in molecular weight decreases drug diffusion rate and therefore drug release rate.

The size of micro-particle also affects the rate of drug release. As the size of micro-particle decreases, the ratio of surface area to volume of the micro-particle increases. Thus, for a given rate of drug diffusion, the rate of drug release from the micro-particle will increase with decreasing micro-particle size. In addition, water penetration into smaller micro-particle may be quicker due to the shorter distance from the surface to the center of the micro-particle.

In addition, the property and amount of drug can also affect the rate of drug release. In an example, the drug powder disclosed herein uses microparticles having sizes between 1 μm and 250 μm, preferably less than 50 μm. The composition of PLGA preferably includes a ratio equal to or more than 50% by weight of polylactic acid (PLA). In one preferred embodiment, each PLGA micro-particle contains 1-50% of drug by weight. Molecular weight of PLGA may be between 7,000 and 150,000 Daltons, preferably 7,000 to 75,000 Daltons.

PLGA Microparticle Fabrication

Microparticles in the present invention can be prepared by microencapsulation, spray drying, precipitation, hot melt microencapsulation, co-extrusion, precision particle fabrication (PPF) or other fabrication techniques. Microencapsulation techniques use single, double or multiple emulsion process in combination with solvent removal step such as evaporation, extraction or coacervation step. They are the most commonly used techniques to prepare micro-particles. The above techniques including the microencapsulation techniques can be used for water soluble drug, organic solvent soluble drug and solid powder drug.

Hydrogel

Hydrogel is a hydrophilic polymer that can swell in water and hold a large amount of water. A three-dimensional structure results from the hydrophilic polymer chains held by crosslinks. The hydrogel is a very good absorbent which can absorb a large amount of water up to more than 10 times its own weight. It is used for many applications such as scaffolds in tissue engineering, sustained drug delivery system, breast implant, wound dressing, disposable diaper and other applications. The hydrogel can be prepared from synthetic polymer or natural polymer. The synthetic polymer includes polyhydroxy ethyl methacrylate (PHEMA), polyethylene glycol (PEG), polyvinyl alcohol (PVA), polyvinylpyrrolidone (PVP), polyimide (PI), polyacrylate (PA), polyurethane (PU) and other synthetic polymers. The natural polymer includes collagen, hyaluronic acid, alginate, chitosan and other natural polymers.

Again, in one embodiment, the present invention uses hyaluronic acid (HA) as its hydrogel component. It is a linear polysaccharide formed from N-acetyl-D-glucosamine and glucuronic acid with a molecular weight ranging from 2×10⁵ to 1×10⁷ daltons. It is naturally abundant in biological fluids and tissues. It is biocompatible, biodegradable, non-immunogenic and non-toxic. HA is used in many clinical applications such as intra-articular injection for treating osteoarthritis patients, wound healing, treating dry eye and other applications. Again, in an example, the drug powder is made by overcoating PLGA-drug microparticles with hyaluronic acid.

The HA-overcoated PLGA drug microparticles disclosed herein have many advantages over non-coated PLGA-drug microparticles. Some of these advantages are improved immunogenicity, potential zero-order drug release and longer drug release time.

HA-PLGA-drug Microparticle Fabrication

In one embodiment, HA-PLGA-drug microparticles are prepared by overcoating PLGA-drug microparticles with HA. First HA may be dissolved in basic aqueous solution. PLGA-drug microparticles may then be suspended in the HA solution by stirring. Then, BDDE (1,4-butanediol diglycidyl ether) may be added as a crosslinker. The resulting solution is then added into an oil like vegetable oil and stirred with a mechanical stirrer. The resulting spherical crosslinked microparticles may be then collected and washed several times with haxane.

In another embodiment, PLGA-drug microparticles may be suspended in an aqueous HA solution. After drying the water by a vacuum oven at around 50° C. the remaining solid can be ground by a ball mill to obtain HA-PLGA-drug microparticles.

Examples of Target Diseases and Drugs that can Benefit from the Disclosed System and Method

For the reasons set forth hereinbefore, getting drugs effectively, efficiently and safely across the blood-brain barrier is critical to developing successful therapies to treat diseases associated with the brain, such as brain cancer (glioblastoma), Alzheimer's disease, Parkinson's disease, amyotrophic lateral sclerosis (ALS) and other neurodegenerative diseases. Glioblastoma (GBM) is the most common and aggressive brain cancer. Current standard treatment is a surgery and then a combination of radiotherapy and temozolomide (TMZ), followed by adjuvant TMZ. TMZ is an alkylating agent which is taken orally. Although TMZ is able to cross the BBB, it is not optimal for treating glioblastoma. TMZ is an alkylating agent which is a conventional chemotherapeutic agent. The conventional chemotherapeutic agent is non-specific to cells, meaning that it kills both cancer cells and normal healthy cells. Such chemotherapeutic agent is very toxic and causes severe side effects. New drugs developed or being developed are more selective to cancer cells and effective against them. The problem is that most of these new drugs do not cross the BBB, thus the need for the disclosed system and method.

Even with aggressive treatments including surgical resection, radiotherapy and chemotherapy, the median length of survival of patients is only 15 months. A complete surgical resection is difficult due to the invasiveness of tumor cells into the surrounding normal brain tissue. Aggressive surgical resection may impair some neurological functions such as vision, speech, etc. Recently there has been a significant understanding of GBM at molecular level such as relating to various gene alterations. Thus, there is a pressing need to move new drugs into the clinic for better treatment. However, due to the shortcomings of the existing drug delivery methods described in the Background section of this application, the BBB still stands as a critical unresolved hurdle for developing effective therapies. Again, these new drugs are more selective to their matching oncogenes than temozolomide or other currently available therapies but for them to be effective they need to be delivered to the diseased tissue successfully. Thus, there is a need for the disclosed system and method that can deliver any class of drugs into the brain successfully. The new drugs are often biologics with a large molecular weight (>180,000 Daltons) which are impossible to deliver effectively through conventional oral and intravenous route. Therefore, the method disclosed herein of effectively, efficiently and safely bypassing the BBB and delivering any class of drugs precisely into the tumor tissue is critical to developing effective GBM therapies.

Alzheimer's disease (AD) is a chronic neurodegenerative disorder associated with accumulation of β-amyloid plaque and intracellular neurofibrillary tangles (tau) in the brain. Approximately 5.5 million people in the US and 44 million people worldwide have AD. AD is the sixth leading cause of death in the US. As of 2019, there are six FDA approved prescription drugs to treat AD. However, these drugs can only relieve symptoms of the disease temporarily (palliative treatment) and none one of them has proven the ability to cure or stop the progression of the disease (disease modifying therapy, DMT). In addition, the efficiency of these drugs varies from person to person and they have some side effects such as nausea, diarrhea and vomiting. Currently there are many new DMT drug developments underway. In order to develop successful therapy of AD, the role of the BBB has to be effectively and safely overcome. Therefore, the method disclosed herein of effectively, efficiently and safely bypassing the BBB and delivering any class of drugs precisely into the tumor tissue is critical to developing effective AD therapies.

Parkinson's disease (PD) is a progressive, long-term neurodegenerative disease that affects mainly movement. There are approximately 10 million people worldwide and 1 million people in the US with PD. Currently, the most effective therapy is using the combination of levodopa and carbidopa but treats only symptoms (palliative treatment). In addition to the levodopa and carbidopa combination, there are many other palliative drugs such as monoamine oxidase B (MAO B) inhibitors, catechol O-methyltransferase (COMT) inhibitors, anticholinergics and amantadine. Currently, there are many new DMT drug developments underway. In order to develop successful therapy of PD, the role of the BBB has to be effectively and safely overcome. Therefore, the method disclosed herein of effectively, efficiently and safely bypassing the BBB and delivering any class of drugs precisely into the tumor tissue is critical to developing effective PD therapies.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims.

If present, use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the aspects, embodiments or examples shown should be considered as exemplars, rather than limitations on the apparatus or procedures disclosed or claimed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

If means-plus-function limitations are recited in the claims, the means are not intended to be limited to the means disclosed in this application for performing the recited function, but are intended to cover in scope any equivalent means, known now or later developed, for performing the recited function.

Claim limitations should be construed as means-plus-function limitations only if the claim recites the term “means” in association with a recited function.

If any presented, the claims directed to a method and/or process should not be limited to the performance of their steps in the order written, and one skilled in the art can readily appreciate that the sequences may be varied and still remain within the spirit and scope of the present invention.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples. Hence, the scope of the invention is defined by the accompanying claims and their equivalents. Further, each and every claim is incorporated as further disclosure into the specification.

What is claimed is:

1. A brain drug delivery system comprising:
a catheter having a first channel with a first exit hole and a second channel with a second exit hole, both the first exit hole and the second exit hole being disposed at a distal end of the catheter, the second exit hole being adapted such that to cause a sideways turn of a movable inner plastic tube exiting therethrough, wherein the sideways turn is of an obtuse angle;
a delivery assembly that fits slidably and selectively into the first and second channel, and that is guidable by an imaging system, the delivery assembly having:
the movable inner plastic tube having a tube exit hole and a sharp-edged tip; and
an image-guided plunger being adapted to penetrate the movable inner plastic tube and push a powder form of drug microparticles via the tube exit hole.

2. The system of claim 1, wherein the powder form of drug microparticles is an hydrogel-PLGA.

3. The system of claim 1, wherein the image guiding system is an ultrasound system.

4. The system of claim 1, wherein the catheter is made of a silicone material.

5. The system of claim 1, wherein the brain drug delivery system is adapted to move rotationally.

6. The system of claim 1, wherein the obtuse angle is within a range of 120-150 degrees.

7. The system of claim 1, wherein the second exit hole is disposed on a side of the catheter.

8. A brain drug delivery system comprising:
a catheter having a first channel with a first exit hole and a second channel with a second exit hole, both the first exit hole and the second exit hole being disposed at a distal end of the catheter, the second exit hole being adapted such that to cause a sideways turn of a movable inner plastic tube exiting therethrough, the catheter being adapted to house a delivery assembly selectively within the first and second channel;
the delivery assembly having:
the movable inner plastic tube having a tube exit hole; and
an image-guided plunger being adapted to penetrate the movable inner plastic tube and push a powder form of drug microparticles via the tube exit hole.

9. The system of claim 8, wherein the image-guided plunger has a tip with a sharp edge.

10. The system of claim 8, wherein the imaging system is magnetic resonance imaging.

11. The system of claim 8, wherein the catheter is made of a latex material.

12. The system of claim 8, wherein the brain drug delivery system is adapted to move both rotationally and up and down.

13. The system of claim 8, wherein the sideways turn is of an obtuse angle.

14. The system of claim 8, wherein the second exit hole is disposed on a side of the catheter.

15. A method of a brain drug delivery system comprising the steps of:
selecting a catheter having a first channel with a first exit hole and a second channel with a second exit hole, both the first exit hole and the second exit hole being disposed at a distal end of the catheter, wherein the second exit hole being adapted such that to cause a sideways turn of a movable inner plastic tube exiting therethrough;
selectively fitting a delivery assembly into the first or second channel of the catheter, the delivery assembly having the movable inner plastic tube having a tube exit hole and an image-guided plunger;
inserting the catheter with the delivery assembly into a subject;
positioning the distal end of the catheter in a proximity of a targeted wound area;
pushing the delivery assembly into the wound area in the subject;
retracting the image-guided plunger;
loading drug particles into the movable inner plastic tube;
reinserting the image-guided plunger into the movable inner plastic tube; and
releasing the drug particles into the wound area either straight via the first exit hole or at an angle via the second exit hole.

16. The method of claim 15, wherein positioning comprises guiding the catheter with the delivery assembly using an imaging system.
